(19) United States
(12) Patent Application Publication
(10) Pub. No.: US 2025/0057488 A1
(43) Pub. Date: Feb. 20, 2025

(54) SYSTEM AND METHOD FOR ALERTING USERS BASED ON BRAIN SIGNALS

(71) Applicant: Mirza Faizan, Irving, TX (US)

(72) Inventors: Mirza Faizan, Irving, TX (US); Nakshatra Piduri, Plano, TX (US); Advaita Piduri, Plano, TX (US); Aarifa Fatima, Patna (IN); Aroush Fatima, Patna (IN); Anushka Sharma, Frisco, TX (US)

(21) Appl. No.: 18/235,853

(22) Filed: Aug. 20, 2023

(51) Int. Cl. A61B 5/00; A61B 5/16; A61B 5/291; A61B 5/377

(52) U.S. Cl. CPC A61B 5/746; A61B 5/165; A61B 5/291; A61B 5/377; A61B 5/6803; A61B 5/7475

(57) ABSTRACT

A system, a method, and a computer program product is provided for alerting users based on brain signals. The alerting system receives brain signals from a wearable device worn by a user. The brain signals correspond to electroencephalography (EEG) signals associated with a brain of the user. The alerting system detects a spike in the received brain signals. The detected spike indicates an increase in a stress level of the user. The alerting system further determines one or more actions to be triggered based on the detected spike in the received brain signals. The alerting system controls a user device to trigger a first action associated with an initiation of a first call, indicating the increased stress level of the user, to a first set of user devices associated with a first set of users. The first action is included in the determined one or more actions.

### TECHNOLOGICAL FIELD

The present disclosure generally relates to an alerting system, and more particularly to a system and method for alerting users based on spikes detected in the brain signals of the users.

### BACKGROUND

Nowadays, crimes such as human trafficking, hostage kidnapping, abduction, gun threats, bank robberies, and the like are on a constant rise due to a variety of reasons such as, but not limited to, financial hardships, unemployment, drug consumption, and lack of education. During these crimes, a victim of the crime is often in a stressful situation, and in the majority of the cases, they do not have any access to their mobile phones to call or alert anyone for help. Even if they have access to their mobile phones, they are not able to call or alert people for help due to fear, or stress.

Conventional techniques for alerting safety authorities or emergency contacts of a user, such as the victim of a crime scenario involves calling or sending messages to the authorities or the emergency contacts to inform them about a current situation of the victim and gather help in overcoming the situation. However, these conventional techniques for alerting lack the ability to send alerts to the authorities or the emergency contacts without a reception of an input from the victim. The alert may be sent only after at least one input is received from the victim via a device such as the mobile phone, to alert the concerned authorities or the emergency contacts. In situations where the victim may not have access to such devices or may not be able to provide inputs on the mobile phone, it may be difficult to call or alert the concerned authorities or the emergency contacts about the current situation of the victim.

### BRIEF SUMMARY

A system, a method, and a computer program product are provided herein that focuses on alerting one or more secondary users based on one or more brain signals received from a user using a wearable device.

In one aspect, an alerting system for alerting users based on brain signals is disclosed. The alerting system includes a processor and a memory coupled to the processor. The memory is configured to store a plurality of processor-executable instructions. The processor is configured to execute the plurality of processor-executable instructions. The processor is configured to receive one or more brain signals from a wearable device worn by a user. The received one or more brain signals correspond to electroencephalography (EEG) signals associated with a brain of the user. The processor is further configured to detect at least one spike in the received one or more brain signals. The detected at least one spike indicates an increase in a stress level of the user. The processor is further configured to determine one or more actions to be triggered based on the detected at least one spike in the received one or more brain signals. The processor is further configured to control a first user device to trigger at least a first action included in the determined one or more actions. The first action is associated with an initiation of a first call to a first set of user devices associated with a first set of users to indicate the increased stress level of the user.

In additional system embodiments, the processor is configured to detect a failure in the initiation of the first call from the first user device to the first set of user devices. The processor is further configured to control the first user device to trigger a second action associated with a transmission of a first message to the first set of user devices. The second action has a second priority value associated with it that is less than a first priority associated with the first action. The transmitted first message includes a timestamp indicative of a time of detection the at least one spike and location information associated with a current location of the user.

In additional system embodiments, the processor is further configured to control the first user device to trigger a third action. The third action is associated with transmission of a second message to a second set of user devices associated with a second set of users. The third action is included in the determined one or more actions.

In additional system embodiments, the processor is further configured to control the wearable device to capture the one or more brain signals of the user. The processor is further configured to calibrate the captured one or more brain signals. The calibration of the one or more brain signals is specific to the user. The processor is further configured to detect the at least one spike in the received one or more brain signals based on the calibration. The at least one spike is detected based on at least one of an audio heard by the user, a visual scene seen by the user, a thought of the user, and or a muscular movement of the user.

In additional system embodiments, the processor is further configured to control a first set of wearable devices to capture physiological information. The physiological information is associated with a set of physiological parameters of the user. The physiological information includes at least one of a blood pressure rate, a body temperature, a heart rate, a blood oxygen level, an eye flicker rate, and a pulse rate. The processor is further configured to determine the one or more actions to be triggered based on the detected spike in the received brain signals and the captured physiological information.

In additional system embodiments, the processor is further configured to render an electronic user interface (UI) on the first user device. The displayed electronic UI includes a set of UI elements. The processor is further configured to receive a set of user inputs via the set of UI elements. The set of user inputs are associated with selection of the first set of users and the one or more actions.

In additional system embodiments, the processor is further configured to compare a value of each of the brain signals with a threshold value stored in a database. The processor is further configured to detect the at least one spike in the received brain signals based on the comparison. The processor is further configured to determine the one or more actions to be triggered based on the detected spike from the database.

In additional system embodiments, the wearable device includes one or more electrodes configured to capture the one or more brain signals of the user.

In additional system embodiments, the wearable device corresponds to at least one of a headset, or an eyeglass. A first electrode is attached to a first temple tip of the eyeglass and a second electrode is attached to a second temple tip of the eyeglass. The first electrode and the second electrode are rotatable around the first temple tip and the second temple tip of the eyeglass.

In additional system embodiments, the wearable device includes a hardware button, and the processor is further configured to receive a first user input via the hardware button included in the wearable device. The first user input corresponds to a selection of the hardware button. The processor is further configured to control the first user device to trigger the first action associated with the initiation of the first call to the first set of user devices based on the reception of the first user input.

In additional system embodiments, the initiated first call is a robo-call, and the user device transmits a pre-recorded message associated with the detection of the increased stress level of the user to the first set of user devices associated with the first set of users.

In another aspect, a method for alerting users based on brain signals is provided. The method includes receiving one or more brain signals from a wearable device worn by a user. The received one or more brain signals corresponds to electroencephalography (EEG) signals associated with a brain of the user. The method further includes detecting at least one spike in the received one or more brain signals. The detected spike indicates an increase in a stress level of the user. The method further includes determining the one or more actions to be triggered based on the detected spike in the received one or more brain signals. The method further comprises includes a first user device to trigger at least a first action associated with an initiation of a first call to a first set of user devices associated with a first set of users. The first call indicates the increased stress level of the user to a first set of user devices associated with a first set of users. The first action is included in the determined one or more actions.

In additional method embodiments, the initiated first call is a robo-call and transmits a pre-recorded message associated with the detection of the increased stress level of the user to the first set of user devices associated with the first set of users.

In additional method embodiments, the method includes detecting a failure in the initiation of the first call from the first user device to the first set of user devices. The method further includes controlling the first user device to trigger a second action associated with a transmission of a first message to the first set of user devices. The second action has a second priority value associated with it that is less than a first priority associated with the first action.

In additional method embodiments, the wearable device corresponds to at least one of a headset, or an eyeglass.

In additional method embodiments, a first electrode is attached to a first temple tip of the eyeglass, and a second electrode is attached to a second temple tip of the eyeglass. The first electrode and the second electrode are rotatable around the first temple tip and the second temple tip of the eyeglass.

In yet another aspect, a wearable device for alerting users based on brain signals is disclosed. The wearable device is worn by a user and includes one or more electrodes detachably attached to the wearable device. The wearable device includes a processor and a memory coupled to the processor. The memory is configured to store a plurality of processor-executable instructions. The processor is configured to execute the plurality of processor-executable instructions. The processor is configured to control the one or more electrodes to capture one or more brain signals of the user. The captured one or more brain signals may correspond to an electroencephalography (EEG) signal associated with a brain of the user. The processor is further configured to detect at least one spike in the received one or more brain signals. The detected at least one spike indicates an increase in a stress level of the user. The processor is further configured to determine one or more actions to be triggered based on the detected at least one spike in the received one or more brain signals. The processor is further configured to trigger at least a first action included in the determined one or more actions. The first action is associated with an initiation of a first call to a first set of user devices associated with a first set of users to indicate the increased stress level of the user.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

Having thus described example embodiments of the disclosure in general terms, reference will now be made to the accompanying drawings, which are not necessarily drawn to scale, and wherein:

FIG. 1 is a block diagram that illustrates an exemplary network environment for alerting users based on brain signals, in accordance with an embodiment of the disclosure.

FIG. 2 is a block diagram of the alerting system of FIG. 1, in accordance with an embodiment of the disclosure.

FIG. 3 is a diagram that depicts an exemplary first electronic user interface (UI) rendered on the user device, in accordance with an embodiment of the disclosure.

FIG. 4 is a diagram that illustrates exemplary operations for alerting users based on brain signals, in accordance with an embodiment of the disclosure.

FIG. 5 is a diagram that illustrates an exemplary alert message on a user device associated with a user, in accordance with an embodiment of the disclosure.

FIG. 6 is a diagram that illustrates an exemplary first message on a first user device associated with a first user of the first set of user devices, in accordance with an embodiment of the disclosure.

FIG. 7 is a diagram that illustrates an exemplary eyeglass, as a wearable device, for alerting users based on brain signals, in accordance with an embodiment of the disclosure.

FIG. 8 is a diagram that illustrates an exemplary eyeglass for alerting users based on brain signals, in accordance with an embodiment of the disclosure.

FIG. 9 is a diagram that illustrates a set of locations on the scalp of the user for the placement of the one or more electrodes, in accordance with an embodiment of the disclosure.

FIG. 10 is a diagram that illustrates an exemplary scenario for alerting users based on brain signals, in accordance with an embodiment of the disclosure.

FIG. 11 is a flowchart that illustrates an exemplary method for alerting users based on brain waves, in accordance with some embodiment of the disclosure.

FIG. 12 is a flowchart that illustrates an exemplary method for alerting users based on brain waves using eyeglass, in accordance with some embodiment of the disclosure.

### DETAILED DESCRIPTION

In the following description, for purposes of explanation, numerous specific details are set forth in order to provide a thorough understanding of the present disclosure. It will be apparent, however, to one skilled in the art that the present disclosure may be practiced without these specific details. In other instances, systems and methods are shown in block diagram form only in order to avoid obscuring the present disclosure.

Some embodiments of the present disclosure will now be described more fully hereinafter with reference to the accompanying drawings, in which some, but not all, embodiments of the disclosure are shown. Indeed, various embodiments of the disclosure may be embodied in many different forms and should not be construed as limited to the embodiments set forth herein; rather, these embodiments are provided so that this disclosure will satisfy applicable legal requirements. Like reference numerals refer to like elements throughout. Also, reference in this specification to “one embodiment” or “an embodiment” means that a particular feature, structure, or characteristic described in connection with the embodiment is included in at least one embodiment of the present disclosure. The appearance of the phrase “in one embodiment” in various places in the specification are not necessarily all referring to the same embodiment, nor are separate or alternative embodiments mutually exclusive of other embodiments. Further, the terms “a” and “an” herein do not denote a limitation of quantity, but rather denote the presence of at least one of the referenced item. Moreover, various features are described which may be exhibited by some embodiments and not by others. Similarly, various requirements are described which may be requirements for some embodiments but not for other embodiments. As used herein, the terms “data,” “content,” “information,” and similar terms may be used interchangeably to refer to data capable of being displayed, transmitted, received and/or stored in accordance with embodiments of the present disclosure. Thus, use of any such terms should not be taken to limit the spirit and scope of embodiments of the present disclosure.

As defined herein, a “computer-readable storage medium,” which refers to a non-transitory physical storage medium (for example, volatile or non-volatile memory device), may be differentiated from a “computer-readable transmission medium,” which refers to an electromagnetic signal.

The embodiments are described herein for illustrative purposes and are subject to many variations. It is understood that various omissions and substitutions of equivalents are contemplated as circumstances may suggest or render expedient but are intended to cover the application or implementation without departing from the spirit or the scope of the present disclosure. Further, it is to be understood that the phraseology and terminology employed herein are for the purpose of the description and should not be regarded as limiting. Any heading utilized within this description is for convenience only and has no legal or limiting effect.

In comparison with the traditional approaches, the present disclosure provides an automatic, efficient, effective, and fast way of alerting authorities and/or emergency contacts about the condition of a user based on the brain activity of the user, and without any dependence on user input(s). The present disclosure may also be able to detect any abnormal stress levels that may be induced in the user and alert the authorities and/or emergency contacts about the induced stress. Unlike the traditional techniques where an access to a mobile phone is required, the present disclosure may be capable of alerting the authorities and/or emergency contacts even when the user has no immediate access to any mobile device.

Furthermore, the present disclosure automatically transmits a text message that may include a timestamp indicative of the time of detection of increased stress level, and the current location of the user. Such information may be vital for providing quick assistance to the user. Also, the present disclosure describes a wearable device with an in-built slot for inserting a subscriber identity module (SIM) card so that the authorities and/or the emergency contacts are alerted even if the mobile device of the user may be placed miles away from the user. In some implementation scenarios, the SIM card may be embedded (or pre-installed) within the wearable device so that the authorities and/or the emergency contacts are alerted without the presence of the mobile device of the user nearby.

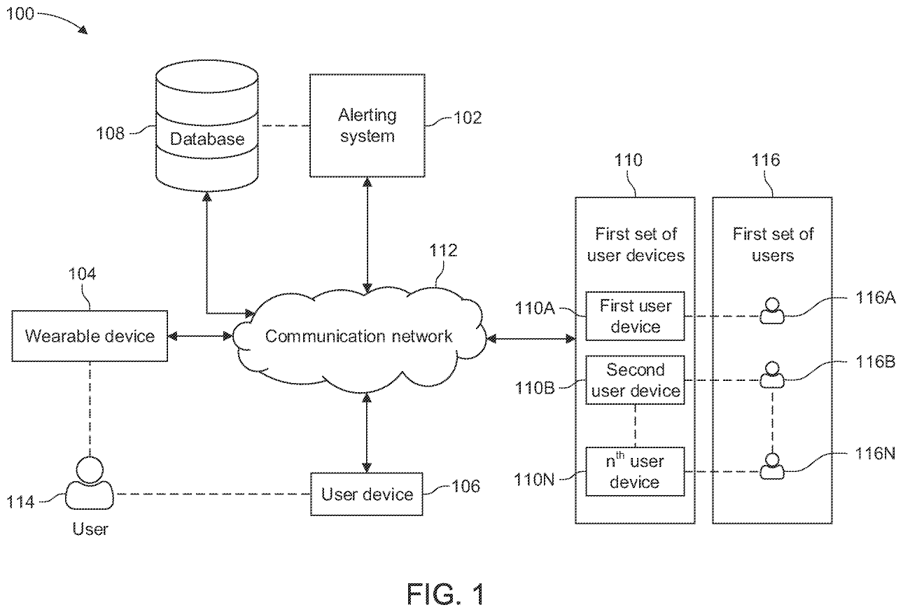

FIG. 1 is a block diagram that illustrates an exemplary network environment for alerting users based on brain signals, in accordance with an embodiment of the disclosure. With reference to FIG. 1, there is shown a network environment 100. The network environment 100 may include an alerting system 102, a wearable device 104, and a user device 106. There is further shown a database 108, a first set of first user devices 110, and a communication network 112. With reference to FIG. 1, there is further shown a user 114 associated with the user device 106, and a first set of users 116 associated with the first set of first user devices 110. The first set of user devices 110 may include a first user device 110A, a second user device 110B, and an Nth user device 110N. The first set of users 116 may include a first user 116A, a second user 116B, and an Nth user 116N. In an embodiment, the first user device 110A may be associated with the first user 116A, the second user device 110B may be associated with the second user 116B, and the Nth user device 110N may be associated with the Nth user 116N.

The alerting system 102 may include suitable logic, circuitry, interfaces, and/or code that may be configured to receive one or more brain signals from the wearable device 104 worn by the user 114. The brain signals may correspond to electroencephalography (EEG) signals associated with a brain of the user 114. The alerting system 102 may be further configured to detect at least one spike in the received one or more brain signals. The detected spike may indicate an increase in a stress level of the user 114. The alerting system 102 may be further configured to determine one or more actions to be triggered based on the detected at least one spike in the received one or more brain signals. The alerting system 102 may further be configured to control the user device 106 to trigger at least a first action associated with an initiation of a first call to the first set of user devices 110 associated with the first set of users 116. The first call may indicate the increased stress level of the user 114 to the first set of users 116. The first action may be included in the determined one or more actions. Examples of such the alerting system 102 may include, but not be limited to, a server, a computing device, a mainframe machine, a computer workstation, a smartphone, a cellular phone, a mobile phone, and a consumer electronic (CE) device.

The wearable device 104 may include suitable logic, circuitry, interfaces, and/or code that may be configured to capture the one or more brain signals associated with a brain of the user 114. The one or more brain signals may correspond to the EEG signals associated with the brain of the user 114. In an embodiment, the wearable device 104 may be worn by the user 114. Specifically, the wearable device 104 may include one or more electrodes that may be configured to capture the one or more brain signals of the user 114. Each of the captured one or more brain signals may correspond to an electrical pattern that may be generated by a synchronized activity of a plurality of neurons in the brain of the user 114. Examples of the wearable device 104 may include, but are not limited to, a headset, an eyeglass, and any other device with the capability to capture the brain signals.

The user device 106 may include suitable logic, circuitry, interfaces, and/or code that may be configured to initiate a robo-call to the first set of user devices 110. The user device 106 may be further configured to receive one or more user input(s) from the user 114. In another embodiment, the user device 106 may be configured to transmit a pre-recorded message associated with the detection of the increased stress level of the user 114 to the first set of user devices 110 and/or a second set of user devices. Examples of the user device 106 may include, but are not limited to, a mobile device, a smartphone, a cell phone, a consumer device, a personal digital assistant (PDA), a computing device, a mainframe machine, a server, a computer workstation, and/or any other device that can initiate calls.

The database 108 may include suitable logic, circuitry, code, and/or interfaces that may be configured to store first information associated with the one or more rules, second information associated with one or more authorities, third information associated with one or more actions, and fourth information associated with the first set of users 116, and/or a second set of users. In another embodiment, the database 108 may store program instructions to be executed by the alerting system 102. Example implementations of the database 108 may include, but are not limited to, a centralized database, a distributed database, a no structured query language (NoSQL) database, a cloud database, a relational database, a network database, an object-oriented database, and a hierarchical database.

Each of the first set of user devices 110 may include suitable logic, circuitry, interfaces, and/or code that may be configured to receive the robo-call that may be initiated from the user device 106. In another embodiment, each of the first set of user devices 110 may be configured to receive the pre-recorded message associated with the detection of the increased stress level of the user 114. In another embodiment, each of the first set of user devices 110 may be configured to receive the first message and/or a second message from the alerting system 102 and render the received first message and/or the second message on a display screen associated with the corresponding user device. Examples of the each of the first set of user devices 110 may include, but are not limited to, a mobile device, a smartphone, a cell phone, a consumer device, a personal digital assistant (PDA), a computing device, a mainframe machine, a server, and a computer workstation.

The communication network 112 may include a communication medium through which the alerting system 102, the wearable device 104, the user device 106, the database 108, and the first set of user devices 110 may communicate with each other. The communication network 112 may be one of a wired connection or a wireless connection. Examples of the communication network 112 may include, but are not limited to, the Internet, a cloud network, a Wireless Fidelity (Wi-Fi) network, a Personal Area Network (PAN), a Local Area Network (LAN), or a Metropolitan Area Network (MAN). Various devices in the network environment 100 may be configured to connect to the communication network 112 in accordance with various wired and wireless communication protocols. Examples of such wired and wireless communication protocols may include, but are not limited to, at least one of a Transmission Control Protocol and Internet Protocol (TCP/IP), User Datagram Protocol (UDP), Hypertext Transfer Protocol (HTTP), File Transfer Protocol (FTP), Zig Bee, EDGE, IEEE 802.11, light fidelity (Li-Fi), 802.16, IEEE 802.11s, IEEE 802.11g, multi-hop communication, wireless access point (AP), device to device communication, cellular communication protocols, and Bluetooth (BT) communication protocols.

In operation, the user 114 may be a victim of a crime or may be performing an activity. Based on the current situation of the user 114, the stress may be inducted in the user 114. The alerting system 102 may be configured to receive the one or more brain signals from the wearable device 104. The wearable device 104 may be worn by the user 114. In an embodiment, the alerting system 102 may be configured to receive the one or more brain signals from the wearable device 104 via the communication network 112. The one or more brain signals may correspond to one or more electrical patterns of activity that occur in the brain of the user 114. Each of the one or more brain signals may correspond to an electrical pattern that may be generated by a synchronized activity of a plurality of neurons in the brain of the user 114, which may communicate with each other through one or more electrochemical impulses. Each brain signal may have a corresponding frequency and amplitude that may vary depending on a mental state of the user 114, such as, but not limited to, concentration, relaxation, or sleep.

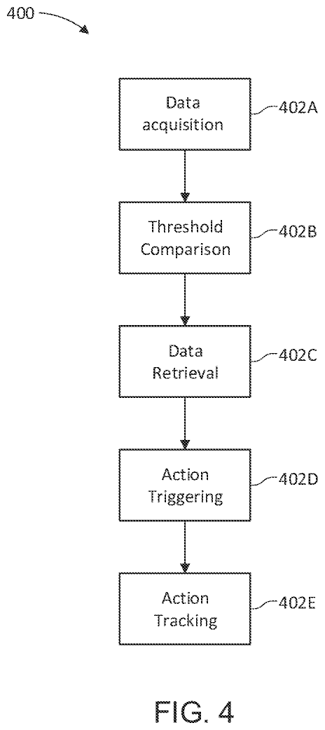

The alerting system 102 may further be configured to detectat least one spike in the received one or more brain signals. The detected at least one spike may be indicative of an increase in a stress level of the user 114. In an embodiment, the at least one spike may be detected based on at least one of an audio heard by the user 114, a visual scene seen by the user 114, a thought of the user 114, and or a muscular movement of the user 114. Details about the detection of the at least one spike are provided, for example, in FIG. 4.

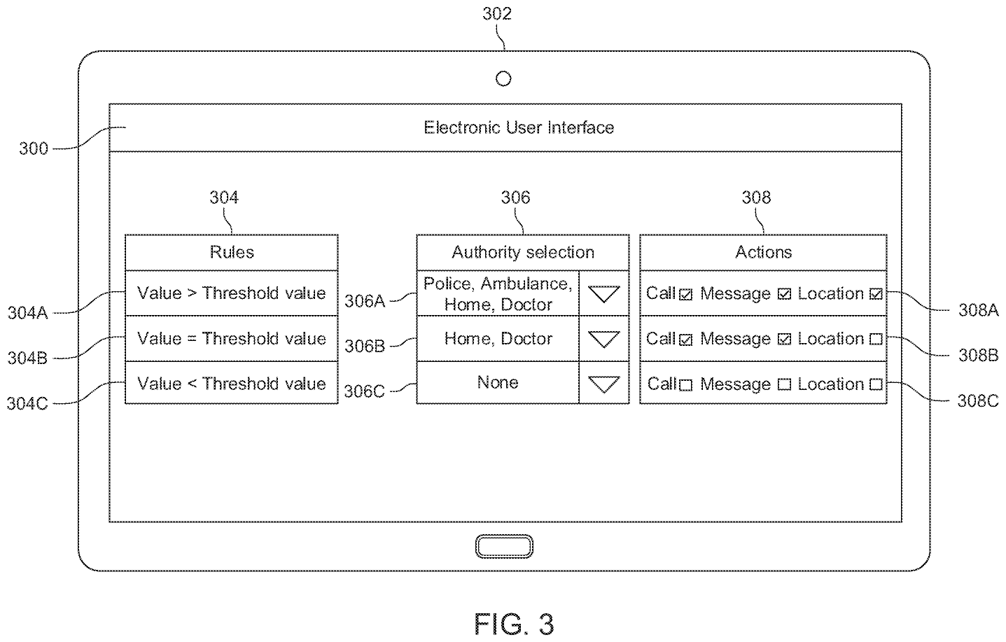

The alerting system 102 may be further configured to determine the one or more actions to be triggered based on the detected at least one spike in the received one or more brain signals. The determined one or more actions may be stored in the database 108. Details about the one or more actions are provided, for example, in FIG. 3.

Based on the determined one or more actions, the alerting system 102 may further be configured to control the user device 106 to trigger a first action of the determined one or more actions. The triggered first action may be associated with an initiation of a call to the first set of user devices 110 thereby alerting the first set of users 116 about the increase in stress levels in the brain of the user 114. Details about the initiation of the first call are provided, for example, in FIG. 4.

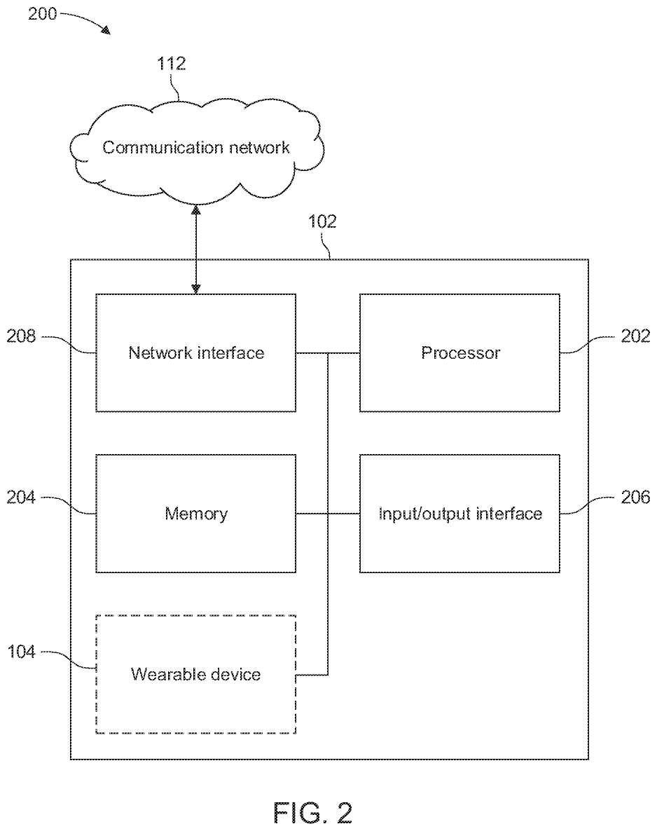

FIG. 2 is a block diagram of the alerting system of FIG. 1, in accordance with an embodiment of the disclosure. FIG. 2 is explained in conjunction with elements from FIG. 1. With reference to FIG. 2, there is shown a block diagram 200 of the alerting system 102. The alerting system 102 may include a processor 202, a memory 204, an input/output interface 206, and a network interface 208. The alerting system 102 may also include the wearable device 104. The processor 202 may be communicatively coupled to the memory 204, the input/output interface 206, the network interface 208, and the wearable device 104.

The processor 202 may include suitable logic, circuitry, and/or interfaces that may be configured to execute program instructions associated with different operations to be executed by the alerting system 102. The processor 202 may be configured to receive the one or more brain signals, detect the at least one spike, determine the one or more actions, and control the user device 106 first set of user devices 110. The processor 202 may include one or more specialised processing units, which may be implemented as an integrated processor or a cluster of processors that perform the functions of the one or more specialized processing units, collectively. The processor 202 may be implemented based on a number of processor technologies known in the art. Examples of implementations of the processor 202 may be an x86-based processor, a Graphics Processing Unit (GPU), a Reduced Instruction Set Computing (RISC) processor, an Application-Specific Integrated Circuit (ASIC) processor, a Complex Instruction Set Computing (CISC) processor, a microcontroller, a central processing unit (CPU), and/or other computing circuits.

The memory 204 may include suitable logic, circuitry, and/or interfaces that may be configured to store program instructions to be executed by the processor 202. The memory 204 may be further configured to store a plurality of processor-executable instructions to be executed by the alerting system 102. The memory 204 may be further configured to store a user profile associated with the user 114 along with a threshold value for an amplitude associated with the one or more brain signals of the user 114. In another embodiment, the memory 204 may be further configured to store the one or more actions to be triggered based on the detection of the at least one spike. Examples of implementations of the memory 204 may include, but are not limited to, Random Access Memory (RAM), Read Only Memory (ROM), Electrically Erasable Programmable Read-Only Memory (EEPROM), Hard Disk Drive (HDD), a Solid-State Drive (SSD), a CPU cache, and/or a Secure Digital (SD) card.

The input/output (I/O) interface 206 may include suitable logic, circuitry, interfaces, and/or code that may be configured to receive an input and provide an output based on the received input. The I/O interface 206 may include various input and output devices, which may be configured to communicate with the processor 202. For example, the alerting system 102 may receive set of user inputs via the I/O interface 206. The I/O interface 206 may comprise various input and output devices, which may be configured to communicate with different operational components of the alerting system 102. Examples of the I/O interfaces 206 may include, but are not limited to, a touch screen, a keyboard, a mouse, a joystick, a microphone, and a display screen.

The network interface 208 may include suitable logic, circuitry, interfaces, and/or code that may be configured to establish communication between the alerting system 102, the wearable device 104, the user device 106, the database 108, and the first set of user devices 110, via the communication network 112. The network interface 208 may be configured to implement known technologies to support wired or wireless communication. The network interface 208 may include, but is not limited to, an antenna, a radio frequency (RF) transceiver, one or more amplifiers, a tuner, one or more oscillators, a digital signal processor, a coder-decoder (CODEC) chipset, a subscriber identity module (SIM) card, and/or a local buffer.

The network interface 208 may be configured to communicate via offline and online wireless communication with networks, such as the Internet, an Intranet, and/or a wireless network, such as a cellular telephone network, a wireless local area network (WLAN), personal area network, and/or a metropolitan area network (MAN). The wireless communication may use any of a plurality of communication standards, protocols and technologies, such as Global System for Mobile Communications (GSM), Enhanced Data GSM Environment (EDGE), wideband code division multiple access (W-CDMA), code division multiple access (CDMA), LTE, time division multiple access (TDMA), Bluetooth, Wireless Fidelity (Wi-Fi) (such as IEEE 802.11, IEEE 802.11b, IEEE 802.11g, IEEE 802.11n, and/or any other IEEE 802.11 protocol), voice over Internet Protocol (VOIP), Wi-MAX, Internet-of-Things (IoT) technology, Machine-Type-Communication (MTC) technology, a protocol for email, instant messaging, and/or Short Message Service (SMS).

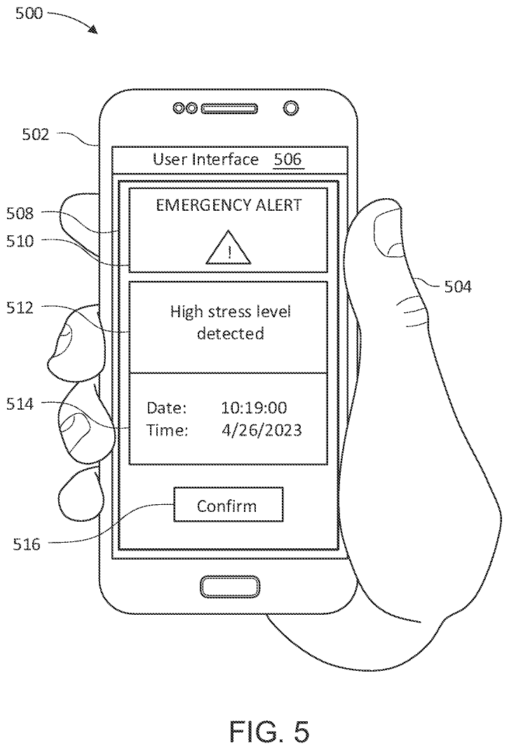

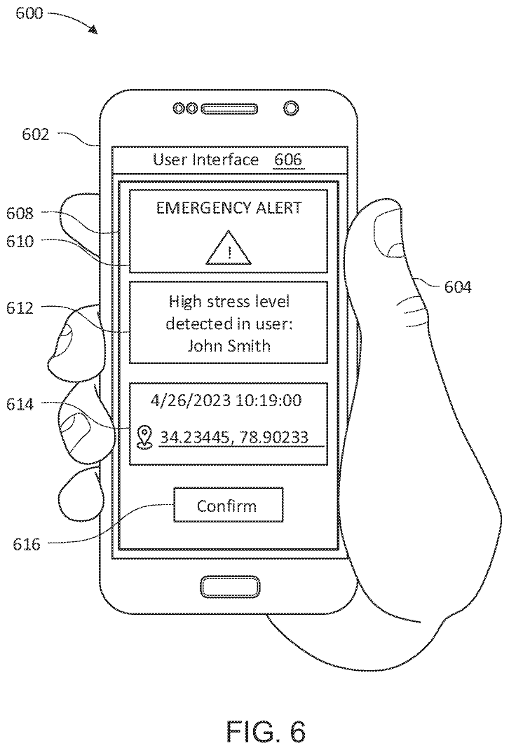

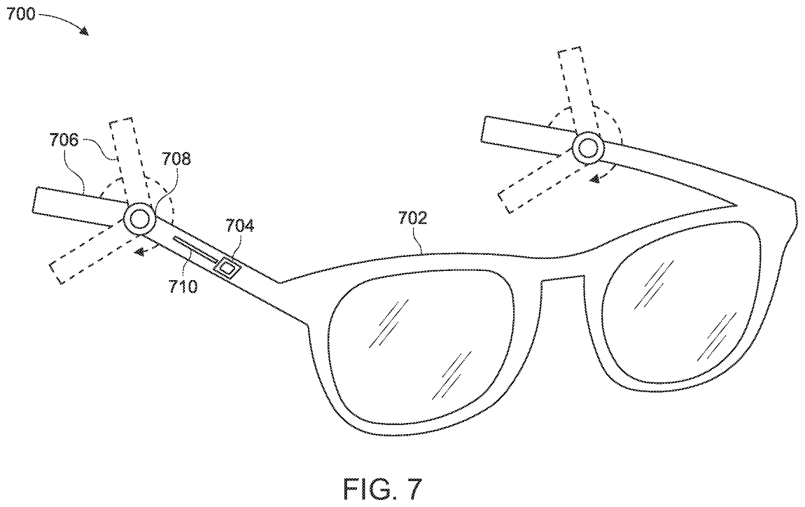

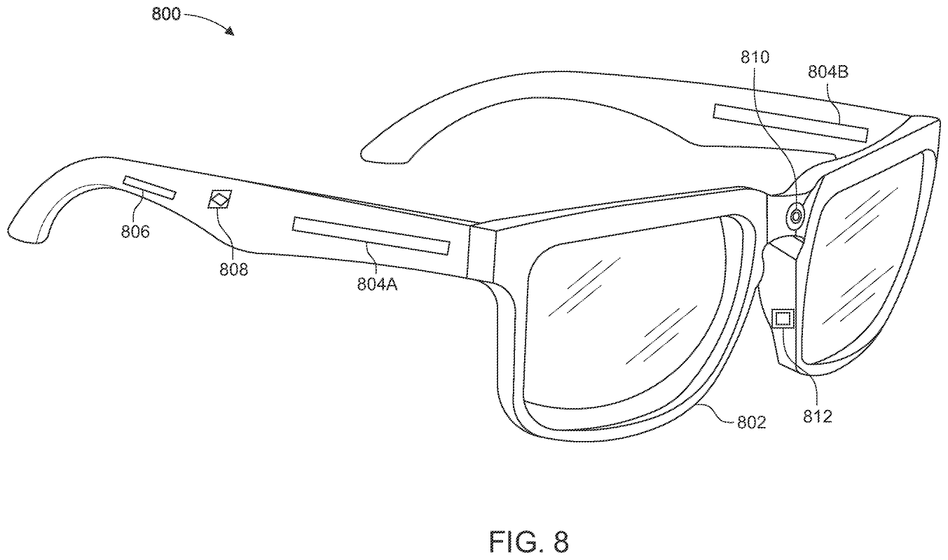

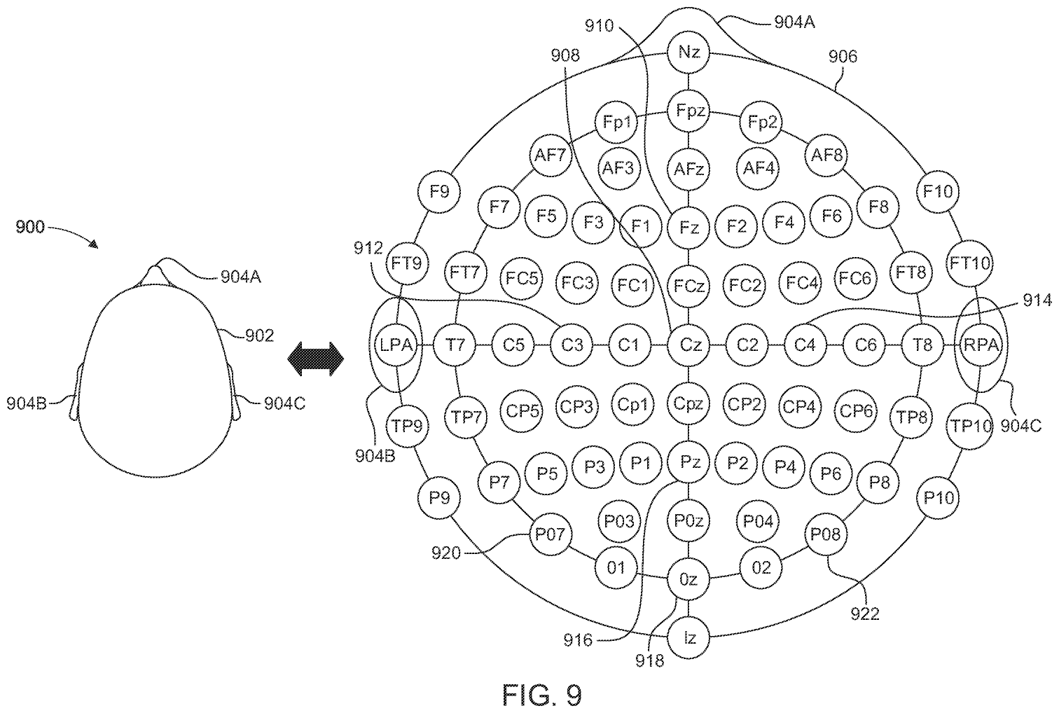

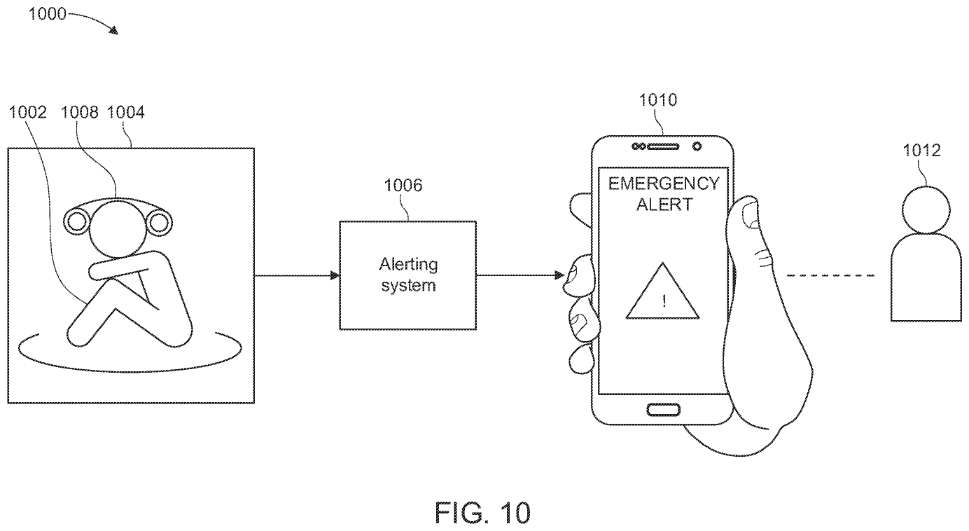

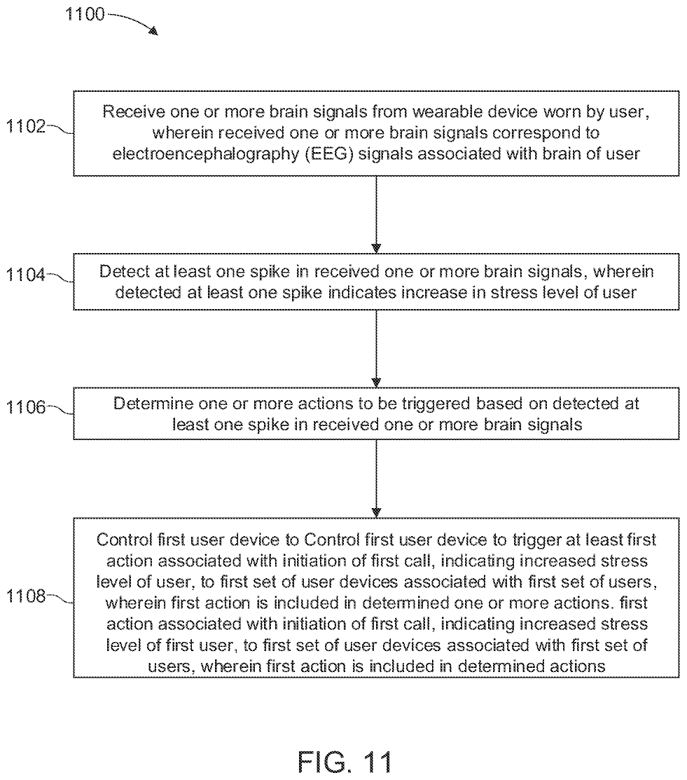

The functions or operations executed by the alerting system 102, as described in FIG. 1, may be performed by the processor 202. Operations executed by the processor 202 are described in detail, for example, in FIG. 3, FIG. 4, FIG. 5, FIG. 6, FIG. 7, FIG. 8, FIG. 9, FIG. 10, and FIG. 11.

FIG. 3 is a diagram that depicts an exemplary first electronic user interface (UI) rendered on the user device, in accordance with an embodiment of the disclosure. FIG. 3 is explained in conjunction with elements from FIG. 1 and FIG. 2. With reference to FIG. 3, there is shown an exemplary electronic UI 300. The electronic UI 300 may be rendered on a user device 302 that may be associated with the user 114 wearing the wearable device 104. The user device 302 may be an exemplary embodiment of the user device 106 of FIG. 1. With reference to FIG. 3, there is further shown a first set of UI elements 304, a second set of UI elements 306, and a third set of UI elements 308.

In an embodiment, the first set of UI elements 304 may be labelled as “Rules” and may include rules for detection of the at least one spike in the one or more brain signals of the user 114. The second set of UI elements 306 may be labelled as “Authorities Selection” and may include one or more authorities who may be informed about the detection of the spike in the one or more brain signals of the user 114. The third set of UI elements 308 may be labelled as “Actions” and may include one or more actions that may be triggered when the rules mentioned in the first set of UI elements 304 are satisfied.

The first set of UI elements 304 may include a first UI element 304A, a second UI element 304B, and a third UI element 304C. Each of the first set of UI elements 304 may correspond to a textbox. The first UI element 304A may correspond to a first rule when a value of an amplitude of at least one brain signal is greater than a threshold value. In such a case, the spike may be detected. The second UI element 304B may correspond to a second rule when the value of an amplitude of at least one brain signal is equal to the threshold value. In such a case, the spike may not be detected. The third UI element 304C may correspond to a third rule when the value of the amplitude of at least one brain signal is less than the threshold value. In such a case, the spike may not be detected.

The second set of UI elements 306 may include a first UI element 306A, a second UI element 306B, and a third UI element 306C. Each of the second set of UI elements 306 may correspond to a textbox. The first UI element 306A may include the first set of users who may be informed about the spike is detected in the brain signals. The second UI element 306B may include a second set of users who may be informed about if the spike is not detected but the value of the amplitude of at least one brain signal is equal to the threshold value. Similarly, the third UI element 306C may include a third set of users who may be informed about if the spike is not detected and the value of the amplitude of at least one brain signal is less than the threshold value.

The third set of UI elements 308 may include a first UI element 308A, a second UI element 308B, and a third UI element 308C. Each of the third set of UI elements 308 may correspond to a textbox and a checkbox. The first UI element 308A may include a first set of actions that may be triggered when the first rule mentioned in the first UI element 304A may be satisfied. The second UI element 308B may include a second set of actions that will be triggered when the second rule mentioned in the second UI element 304B may be satisfied and the third UI element 308C may include a third set of actions that will be triggered when the third rule mentioned in the third UI element 304C may be satisfied.

According to some embodiments of the disclosure, the set of actions may include, but not be limited to, a first action associated with an initiation of a robo-call indicating the increased stress level of the user 114 to the first set of user devices 110 associated with the first set of users 116, and a second action associated with a transmission of a first message or a second message to the first set of user devices 110 associated with the first set of users 116. In another embodiment, the set of actions may also include a third action associated with a transmission of a second message to the second set of user devices associated with the second set of users. Further, the transmitted message may include a time stamp indicative of a time of detection of the at least one spike in the one or more brain signals of the user 114 and location information associated with a current location of the user 114.

As an exemplary scenario, if the user 114 is involved in a car accident and is severely injured. The alerting system 102 may receive the one or more brain signals from the wearable device 104 worn by the user 114. The alerting system 102 may compare the amplitude of each of the received one or more brain signals with the threshold value. The alerting system 102 may further detect the spike in the received one or more brain signals which may indicate an increase in the stress level of the user 114 based on the comparison. According to the rules configured in the first set of UI elements 304, the authorities selected in the second set of UI elements 306 and the actions configured in the third set of elements 308 as shown in FIG. 3, the alerting system 102 may determine a first action to be triggered. The first action may be associated with an initiation of a robo-call indicating the increased stress level of the user 114 to the first set of user devices 110 associated with the first set of users 116 which may include “Police”, “Ambulance”, “Home” and “Doctor”. The second action may be associated with a transmission of a message to the first set of user devices 110 associated with the first set of users 116 which may include “Police”, “Ambulance”, “Home” and “Doctor”. Further, the transmitted message may include a time stamp indicative of a time of detection of the spike in the one or more brain signals of the user 114 and location information associated with the current location of the user 114.

In another scenario, if the spike is not detected but the amplitude of the received one or more brain signals is equal to the threshold value, the second rule mentioned in the second UI element 304B of the first set of UI elements 304 may be satisfied. In such a scenario, the alerting system 102 may be configured to determine the one or more actions to be performed as per the actions selected at the second UI element 308B of the third set of UI elements 308. Specifically, the one or more actions may include control the user device 302 to initiate a call and transmit the message to the authorities (i.e., Home and Doctor) selected at the second UI element 306B of the second set of UI elements 306. Further, the transmitted message may include the time stamp indicative of the time of detection of the spike in the one or more brain signals of the user 114 and may not include the location information associated with the current location of the user 114.

It may be noted that the user device 302 may not be associated with the user 114. In some embodiment, the user device 302 may be associated with one or more family members of the user 114. In another embodiment, all the selections done on the electronic UI 300 may be done by the user 114 or any other person (such as a family member of the user 114 or an administrator). The information related to the one or more rules may be stored as the first information in the database 108. The information related to the selected authorities may be stored as the second information in the database 108. Similarly, information related to the selected one or more actions may be stored as the third information in the database 108.

FIG. 4 is a diagram that illustrates exemplary operations for alerting users based on brain signals, in accordance with an embodiment of the disclosure. FIG. 4 is explained in conjunction with elements from FIG. 1, FIG. 2, and FIG. 3. With reference to FIG. 4, there is shown a block diagram 400 that illustrates exemplary operations from 402A to 402E, as described herein. The exemplary operations illustrated in the block diagram 400 may start at 402A and may be performed by any computing system, apparatus, or device, such as by the alerting system 102 of FIG. 1 or the processor 202 of FIG. 2. Although illustrated with discrete blocks, the exemplary operations associated with one or more blocks of the block diagram 400 may be divided into additional blocks, combined into fewer blocks, or eliminated, depending on the particular implementation.

In an embodiment, the alerting system 102 may operate in two phases-a setup phase and an operational phase. In the setup phase, the alerting system 102 may be configured by the user 114 as described in FIG. 3. The user 114 may use the electronic UI 300 to select the one or more rules, the one or more authorities, and the one or more actions to be performed when the at least one spike is detected in the one or more brain signals of the user 114. The user 114 may further customize a pre-recorded message to be sent to the first set of user devices 110 and/or the second set of users devices. The database 108 may be configured to store the data entered by the user 114 using the electronic UI 300. After the configuration, the alerting system 102 may operate in the phase. In the operational phase, the operations from 402A to 402E may be performed.

At 402A, a data acquisition operation is performed. In the data acquisition operation, the processor 202 may be configured to receive the one or more brain signals associated with the brain of the user 114. The one or more brain signals may be received from the wearable device 104 that may be worn by the user 114. In an embodiment, the processor 202 may be configured to control the wearable device 104 to capture the one or more brain signals associated with the brain of the user 114. The captured one or more brain signals may correspond to an electroencephalography (EEG) signal that may be associated with the brain of the user 114.

In an embodiment, the wearable device 104 may correspond to one of a headset, or an eyeglass and may include one or more electrodes that may be configured to capture the one or more brain signals of the user 114. Each of the one or more electrodes may correspond to a metal disc that may be attached to a scalp of the user 114. Each of the one or more electrodes may be used to measure the electrical activity in the brain, as the one or more brain signals. Each of the one or more electrodes may be one of a surface electrode or an intracranial electrode. The surface electrode may be placed on the scalp and may be non-invasive whereas the intracranial electrode may be placed inside the skull of the user 114 and may be invasive. Details about the wearable device and the one or more electrodes are provided, for example, in FIG. 7.

The wearable device 104 may be further configured to transmit the captured one or more brain signals to the alerting system 102 via the communication network 112. Each of the one or more brain signals may be an electrical signal that may be generated by the brain by a collective activity of the plurality of neurons of the human nervous system. In the human body, the electrical signals may be generated by the flow of charged ions (such as sodium, potassium, and calcium) across the cell membranes of neurons. Such signals are measured using electroencephalography (EEG), which may be a method to record an electro gram of the spontaneous electrical activity of the brain. In one or more embodiments, each of the one or more brain signals may be visualized as a graph between a voltage versus and a time graph.

In an embodiment, the processor 202 may be further configured to calibrate the captured one or more brain signals by analysing one or more parameters of the captured one or more brain signals. The one or more parameters may include, but are not limited, to a sampling rate, a sensitivity, an amplifier integrity, and a frequency. The calibration of the one or more brain signals may determine the threshold value of the amplitude of the brain signals. The determined threshold value may be specific to the user 114 and may change from user to user.

The processor 202 may be further configured to store information associated with the calibrated one or more brain signals in the database 108. The data may be stored in the database 108 using methods that may include, but not be limited to a structured query language (SQL) queries and an insertion of data into a non-structured query language (NOSQL) database.

In another embodiment, the user 114 may be wearing a first set of wearable devices including the wearable device 104. The alerting system 102 may be configured to control the first set of wearable devices to capture physiological information associated with a set of physiological parameters of the user 114. In an embodiment, the first set of wearable devices may include, but are not limited to, a blood pressure rate sensor, a body temperature sensor, a heart rate sensor, a blood oxygen level sensor, and an eye flicker sensor.

In an embodiment, the set of physiological parameters may include, but are not limited to, a blood pressure rate, a body temperature, a heart rate, a blood oxygen level, and an eye flicker rate. The blood pressure rate may correspond to a measure of a force with which the heart pumps blood around the body of the user 114. The body temperature may correspond to a measure of a temperature of the body of the user 114. The heart rate may correspond to a measure of a number of times the heart beats within a certain time period, usually a minute. The blood oxygen level may correspond to a measure of an amount of oxygen circulating in the blood of the user 114. The eye flicker rate may correspond to a measure of a number of times the user 114 may blink at least one eye in a minute.

In an embodiment, the alerting system 102 may be further configured to calibrate the captured physiological information. The alerting system 102 may be further configured to the captured physiological information in the database 108. Similar to the calibration of the one or more brain signals, the alerting system 102 may be configured to calibrate the captured physiological information to determine a threshold value corresponding to each of the set of physiological parameters. This may be done to detect the spike in the one or more brain signals and to further detect the increase in the stress level of the user 114.

At 402B, a spike detection operation is performed. In the spike detection operation, the processor 202 may be configured to detect at least one spike in the received one or more brain signals. The spike may indicate an increase in a stress level of the user 114. The spike in brain signals may refer to a sudden increase in neural activity, which may be measured using the EEG or other neuro-imaging techniques. Such spikes may occur for a variety of reasons, including in response to sensory input, during cognitive processing, or as a result of a pathological conditions such as epilepsy. In another embodiment, the at least one spike may be detected based on at least one of an audio heard by the user 114, a visual scene seen by the user 114, a thought of the user 114, and or a muscular movement of the user 114.

In an embodiment, the detection of the at least one spike in the one or more brain signals may be a complex and nuanced process. In an embodiment, the at least one spike may be detected by looking for short and high-amplitude peaks in the one or more brain signals. The amplitude may refer to a height of a brain signal from the zero point to its peak and may be typically measured in volts. The peak may refer to a local maximum in the amplitude of a signal. It is the highest point of a signal, which may occur only once or multiple times.

The at least one spike in the one or more signals may be detected to identify an increase in the stress levels of the user 114. In an embodiment, the alerting system 102 may be configured to detect the at least one spike using one or more methods. The one or more methods may include, but not be limited to, a threshold comparison method, a waveform template matching method, a principal component analysis (PCA), an independent component analysis (ICA) method and a machine learning (ML) method.

In the threshold comparison method, the processor 202 may be configured to compare a value of each of the detected peak with a threshold value that may be stored in the database 108. The processor 202 may be configured to fetch the threshold value from the database 108. The processor 202 may be further configured to compare each amplitude value of the detected peak with the threshold value. In one embodiment, the spike may be labeled “low” if the amplitude is less than the threshold value. In such a scenario, the spike may not be detected. In another embodiment, the spike may be labeled “medium” if it is equal to the threshold value. In such a scenario, the spike may not be detected. In one embodiment, the spike may be labeled “high” if it is greater than the threshold value. In such a scenario, the spike may be detected.

At 402C, a data retrieval operation is performed. In the data retrieval operation, the processor 202 may be configured to fetch data from the database 108 based on the rules, authority selection, and actions configured using the user interface (UI) of the user device 106, as described in FIG. 3.

The processor 202 may be configured to fetch the data associated with the first set of user devices 110 and/or a second set of user devices based on the detected at least one spike. The data may include a name and contact information associated with the first set of users 116 and/or a second set of users and the first set of user devices 110 and/or the second set of user devices. In an embodiment, the data may further include a pre-recorded message to be transmitted to the first set of user devices 110 and/or the second set of users.

The alerting system 102 may be further configured to determine the one or more actions to be triggered based on the detection of at least one spike in the received one or more brain signals. The alerting system 102 may be configured to determine the one or more actions from the database 108. As discussed above, the one or more actions to be triggered may be provided by the user 114 via the third set of UI elements 308.

In general, the data may be fetched from a database using a query language such as structured query language (SQL) or a programming language that has database access libraries. The general steps involved in fetching data from a database may include, but not be limited to an establishment of a connection with the database, a construction of a query to retrieve the data, an execution of the query, a retrieval of the data, and a processing of the retrieved data.

At 402D, an action triggering operation is performed. In the action triggering operation, the processor 202 may be configured to trigger at least a first action of the one or more actions determined at 402C. The first action may have a first priority value associated with the first action. The first priority value may be the highest priority value among the determined one or more actions.

In an embodiment, the alerting system 102 may be configured to control the user device 106 to trigger at least the first action associated with an initiation of the first call to the first set of user devices 110 associated with the first set of users 116. In an embodiment, the first call may be indicative of the increased stress level of the user 114. The initiated first call may be a robo-call and may transmit a pre-recorded message associated with the detection of the increased stress level of the user 114 to the first set of user devices 110 associated with the first set of users 116.

In an embodiment, a robo-call may be an automated phone call that may deliver a pre-recorded message to the recipient. The first call may be made using an auto dialer, which may be a computer program that can call a large number of phone numbers associated with the first set of user devices 110 in a short period of time. The pre-recorded message may be a message that has been previously recorded about the increase in the stress level of the user 114 and is played back to the recipient as soon as the call is picked up by the first set of users 116.

At 402E, an action tracking operation may be performed. In the action tracking operation, the processor 202 may be configured to track a completion of the first action. In an embodiment, the first action is completed (i.e., the first call is successfully established), then the control may pass to end.

In another embodiment, the processor 202 may be configured to detect a failure in the initiation of the first call from the user device 106 to the first set of user devices 110. The processor 202 may be further configured to control the user device 106 to trigger a second action. The second action may be associated with a transmission of a first message to the first set of user devices 110. In an embodiment, the second action may have a second priority value associated with the second action which may be less than a first priority value associated with the first action. The transmitted first message may include a timestamp that may be indicative of a time of detection the at least one spike in the one or more brain signals. In another embodiment, the transmitted first message may further include location information associated with a current location of the user 114. In an embodiment, the location information may include a pair of coordinates associated with the current location of the user 114. The location information may be vital for quickly helping the user 114.

In another embodiment, the processor 202 may be configured to detect a failure in triggering the second action. In such embodiment, the processor 202 may be configured to control the user device 106 to trigger a third action of the determined one or more actions. The third action may be associated with a transmission of a second message to a second set of user devices associated with a second set of users. In an embodiment, the transmitted second message may include the timestamp that may be indicative of the time of detection the at least one spike in the one or more brain signals. In an embodiment, the first message and the second message may be transmitted through a variety of channels including, but not limited to, a text message, a multimedia message, a voicemail, an email, and a notification.

In an alternate embodiment, the processor 202 may be configured to capture the one or more brain signals even after the detection of the at least one spike. In certain scenarios such as in an accident, the wearable device 104 worn by the user 114 may be disengaged from the user 114 (or may fall off from the body of the user 114). In such scenario, the wearable device 104 may not be able to capture and transmit the one or more brain signals until the user 114 again wears the wearable device 104. In such scenarios if the processor 202 does not receive the one or more brain signals for a pre-specific time period, then the processor 202 may be configured to automatically trigger the first action and/or the second action and/or the third action.

By way of an example and not limitation, in case due to any reason if the wearable device 104 falls off from the user 114 after detection of the at least one spike in the one or more brain signals e.g. after an accident, then the alerting system 102 may be auto activated to trigger the first action and/or the second action and/or the third action if the alerting system 102 does not receive any other brain signal from the wearable device 102 within 10 seconds of the last detected spike.

FIG. 5 is a diagram that illustrates an exemplary alert message on a user device associated with a user, in accordance with an embodiment of the disclosure. FIG. 5 is explained in conjunction with elements from FIG. 1, FIG. 2, FIG. 3, and FIG. 4. With reference to FIG. 5, there is shown a diagram 500 of a user device 502 held by a user 504. The user device 502 may be an exemplary embodiment of the user device 106. The user device 502 may render an electronic user interface (UI) 506. An alert message 508 may be displayed on the user device 502 via the electronic UI 506. The alert message 508 may include a first UI element 510, a second UI element 512, a third UI element 514, and a fourth UI element 516.

The alert message 508 may be displayed on the user device 502 after the detection of the at least one spike in the one or more brain wave signals. As discussed above, the alert message 508 may include the first UI element 510 which may display an alert icon to the user 504. The second UI element 512 may be a textbox and may display a warning related to detection of a high level of stress. The third UI element 514 may be a textbox and may display a timestamp indicative of the time and date of detection of at least one spike in the one or more brain signals of the user 504. The fourth UI element 516 may be a button. Upon the selection of the fourth UI element 516 by the user 504, it may be deemed that the user 504 has read the alert message 508 displayed on the electronic UI 506 of the user device 502.

In an embodiment, the user device 502 may be configured to display the alert message 508 along with an audio alert that may be outputted through one or more speakers installed in the user device 502. In another embodiment, the user device 502 may further be configured to display the alert message 508 without an audio alert that may be outputted through one or more speakers installed in the user device 502. In another embodiment, information associated with the set of physiological parameters may also be rendered on the electronic UI 506 of the user device 502.

FIG. 6 is a diagram that illustrates an exemplary first message on a first user device associated with a first user of the first set of user devices, in accordance with an embodiment of the disclosure. FIG. 6 is explained in conjunction with elements from FIG. 1, FIG. 2, FIG. 3, FIG. 4, and FIG. 5. With reference to FIG. 6, there is shown a diagram 600 of a first user device 602 of the first set of users devices held by a first user 604 of the first set of users. The first user device 602 may be an exemplary embodiment of the first user device 110A. The first user device 602 may render an electronic user interface (UI) 606. A first message 608 may be displayed on the first user device 602 via the electronic UI 606. The first message 608 may include a first UI element 610, a second UI element 612, a third UI element 614, and a fourth UI element 616.

The alerting system 102 may be configured to receive the one or more brain signals from the wearable device 104 worn by the user 114. The received one or more brain signals may correspond to the EEG signals associated with the brain of the user 114. The alerting system 102 may be further configured to detect the at least one spike in the received one or more brain signals. The alerting system 102 may be further configured to determine one or more actions to be triggered based on the detected at least one spike in the received one or more brain signals. The alerting system 102 may be further configured to control the user device 106 to trigger at least a second action associated with the transmission of the first message 608 to the first set of user devices 110.

The first message 608 may be displayed on the first user device 602 after the detection of the at least one spike in the one or more brain wave signals. As discussed above, the first message 608 may include the first UI element 610 which may display an alert icon to the first user 604. The second UI element 612 may be a textbox and may display a warning related to the detection of a high level of stress in the user 114. In an embodiment, the second UI element 612 may further include an identifier (such as a name) associated with the user 114. The identifier may be used to identify the user 114 whose stress level may have increased. The third UI element 614 may be a textbox and may display a timestamp indicative of the time and date of detection of at least one spike in the one or more brain signals of the user 114. In an embodiment, the third UI element 614 may also include location information associated with a current location of the user 114. The fourth UI element 616 may be a button. Upon the selection of the fourth UI element 616 by the first user 604, it may be deemed that the first user 604 has read the first message 608 displayed on the electronic UI 606 of the first user device 602 and notified about the increased stress level of the first user 604.

In an embodiment, the first user 604 may be configured to display the first message 608 along with an audio alert that may be outputted through one or more speakers installed in the first user device 602. In another embodiment, the first user device 602 may be further configured to display the first message 608 without an audio alert that may be outputted through one or more speakers installed in the first user device 602. In another embodiment, information associated with the set of physiological parameters may also be rendered on the electronic UI 606 of the first user device 602.

FIG. 7 is a diagram that illustrates an exemplary eyeglass, as a wearable device, for alerting users based on brain signals, in accordance with an embodiment of the disclosure. FIG. 7 is explained in conjunction with elements from FIG. 1, FIG. 2, FIG. 3, FIG. 4, FIG. 5, and FIG. 6. With reference to FIG. 7, there is shown a diagram 700 that illustrates an exemplary eyeglass 702. The eyeglass 702 may be an exemplary embodiment of the wearable device 104. With reference to FIG. 7, there is further shown a hardware button 704, one or more electrodes 706, one or more temple tips 708, and a slot 710.

In an embodiment, the eyeglass 702 may include the hardware button 704. In an embodiment, the alerting system 102 may be configured to receive a first user input via the hardware button 704 that may be included in the eyeglass 702. The first user input may correspond to a selection of the hardware button 704. The alerting system 102 may be further configured to control the user device 106 to trigger the first action associated with the initiation of the first call to the first set of user devices 116 based on the reception of the first user input.

In an embodiment, the eyeglass 702 may further include one or more electrodes 706 that may be attached to one or more temple tips 708 of the eyeglass 702. Each temple tip of the one or more temple tips 708 may refers to an arm of the eyeglass 702 that may rest behind the ears of a person wearing the eyeglass 702 and may be made of a soft and flexible material such as, but not limited to, a silicone or an acetate.

In some embodiments, each of the one or more electrodes 706 may be detachable to allow the eyeglass to be used as normal eyeglasses. In some embodiments, a first electrode of the one or more electrodes 706 may be attached to a first temple tip of the one or more temple tips 708. Similarly, a second electrode of the one or more electrodes 706 may be attached to a second temple tip of the one or more temple tips 708.

In another embodiment, each of the one or more electrodes 706 may be rotatable in a 180-degree plane around the corresponding temple tip. In another embodiment, each of the one or more electrodes 706 may be configured to measure an electrical signal from the skin surface of the user 114 using the electric potential difference in the body and the one or more electrodes 706, as described in FIG. 4.

In an embodiment, the eyeglass 702 may be configured to support wireless charging to charge the one or more electrodes 706. The eyeglass 702 may include one or more coils that support the wireless charging. Such one or more coils may be made of a conductive material such as a copper wire and may be wound into a specific shape to form the coil.

In another embodiment, the eyeglass 702 may include a slot 710 for inserting a subscriber identity module (SIM) card in the eyeglass 702. In an alternate embodiment, the SIM card may be embedded within the eyeglass 702. In such a scenario, the alerting system 102 may be able to initiate the robo-call from the eyeglass 702 based on the detection of the spike in the one or more brain signals, thereby removing the necessity of the user device 106 for calling purposes. In another embodiment, the alerting system 102 may be able to transmit a first message and/or the second message from the eyeglass 702 to the first set of user devices 116 and/or the second set of user devices based on the detection of the spike in the one or more brain signals, thereby removing the necessity of the user device 106 for messaging purposes.

In another embodiment, a display screen may be integrated within the eyeglass 702. Specifically, the display screen may be integrated with one or more lenses of the eyeglass 702 and may be visible only to the user 114 wearing the eyeglass 702. The display screen may be configured to be controlled based on a movement of at least one eye of the user 114. In an embodiment, the display screen may include one or more options to trigger at least one action of the set of actions. The one or more options may be selected based on the movement of the at least eye of the user 114. By way of example and not limitation, the eyeglass 702 may be configured to initiate the robo-call from the eyeglass 702 based on the movement of at least one eye of the user 114.

FIG. 8 is a diagram that illustrates an exemplary eyeglass for alerting users based on brain signals, in accordance with an embodiment of the disclosure. FIG. 8 is explained in conjunction with elements from FIG. 1, FIG. 2, FIG. 3, FIG. 4, FIG. 5, FIG. 6, and FIG. 7. With reference to FIG. 8, there is shown a diagram 800 that illustrates an exemplary eyeglass 802. The eyeglass 802 may be an exemplary embodiment of the wearable device 104 of FIG. 1. With reference to FIG. 8, there is further shown a first electrode slot 804A, a second electrode slot 804B, a subscriber identity module slot 806, a hardware button 808, an image capture device 810, an audio capture device 812, and an audio rendering device 814.

In an embodiment, the first electrode slot 804A and second electrode slot 804B may correspond to a designated location or space in the eyeglass 802 where a first electrode and a second electrode (not shown) of the one or more electrodes may be inserted or placed. Each of the one or more electrodes may be detachably attached to the corresponding electrode slot. For example, the first electrode slot 804A may hold the first electrode of the one or more electrodes and the second electrode slot 804B may hold the second electrode of the one or more electrodes.

The SIM card slot 806 may correspond to a small slot or compartment on the eyeglass 802 where the user 114 may be able to insert a SIM card for triggering at least the first action of the set of actions. In an embodiment, the eyeglass 802 may be configured to receive a first user input via the hardware button 808 that may be included in the eyeglass 802. The first user input may correspond to a selection of the hardware button 804. The alerting system 102 may be further configured to trigger a first action associated with the initiation of the first call to the first set of user devices 116 based on the reception of the first user input.

The image capture device 810 may include suitable logic, circuitry, and interfaces that may be configured to capture an image of a scene within the field-of-view (FoV) of the image capture device 810. The image capture device 110 may be disposed on a bridge of the eyeglass 802. Examples of the image capture device 810 may include, but are not limited to, an image sensor, a charge-coupled device (CCD), a wide-angle camera, an action camera, a closed-circuit television (CCTV) camera, a camcorder, a digital camera, camera phones, a time-of-flight camera (ToF camera), a night-vision camera, a 360-degree camera, and/or other image capturing devices.

The audio capture device 812 may include suitable logic, circuitry, and/or interfaces that may be configured to capture an audio signal from the user 114 who may be wearing the eyeglass 802. The audio capture device 812 may be further configured to convert the captured audio signal into an electrical signal. Examples of the audio capture device 812 may include, but are not limited to, a recorder, an electret microphone, a dynamic microphone, a carbon microphone, a piezoelectric microphone, a fiber microphone, a (micro-electro-mechanical systems) MEMS microphone, or other microphones are known in the art.

The audio rendering device 814 may include suitable logic, circuitry, and interfaces that may be configured to reproduce or playback an audio of at least one of the first set of users 116. Examples of the audio rendering device 814 may include, but are not limited to, a loudspeaker, a wireless speaker, and/or other computing device with audio reproduction capabilities.

In operation, the user 114 may be wearing the eyeglass 802 and may be a victim of a crime or may be performing an activity. The user may detach the first electrode from the first electrode slot 804A and the second electrode from the second electrode slot 804B and place the first electrode and the second electrode in any two positions of a set of locations on the scalp of the user 114. Details about the set of locations are provided, for example, in FIG. 9.

Once placed on the scalp, the eyeglass 802 may be configured to control the one or more electrodes to capture one or more brain signals of the user 114. In some other embodiment, the wearable device 802 may receive the one or more brain signals from the one or more electrodes as soon as the one or more electrodes are placed on the scalp of the user 114. As discussed above, the captured one or more brain signals may correspond to an electroencephalography (EEG) signal associated with a brain of the user 114. The eyeglass 802 may be further configured to detect at least one spike in the captured one or more brain signals. The detected at least one spike may indicate an increase in a stress level of the user 114. Details about the detection of the at least one spike are provided, for example, in FIG. 3.

The eyeglass 802 may be further configured to determine one or more actions to be triggered based on the detected at least one spike in the received one or more brain signals. The eyeglass 802 may be further configured to trigger at least a first action associated with an initiation of a first call, indicating the increased stress level of the user 114, to a first set of user devices associated 110 with the first set of users 116. The first action may be included in the determined one or more actions.

In an embodiment, the user 114 may be able to communicate with at least one of the first set of uses 116 via the audio capture device 812 and the audio rendering device 814 disposed on the eyeglass 802 and inform the first set of users 116 about their current situation. Moreover, the user 116 may be able to stream the scene in the FoV of the user 114 to at least one of the first set of users 116 via the image capture device 810. Therefore, the user 114 may be quickly able to alert the first set of users 116, and the streaming of the scene that may help the first set of users 116 to quickly identify the user 114 and assist them accordingly. Therefore, the disclosed eyeglass 802 may be capable of quickly assisting users who may be in trouble. Furthermore, the disclosed eyeglass 802 may be disguised as a normal eyeglass to the criminal and thereby not alerting the criminal. Also, the disclosed eyeglass 802 may work standalone and alert the first set of users 116 without any dependency on a mobile device or any other electronic device. Furthermore, the disclosed eyeglass 802 may be capable of operating in remote areas with the SIM (or an in-built SIM) card.

In an embodiment, the eyeglass 802 may include a processor and a memory. The memory may be communicatively coupled to the processor and may store a plurality of processor-executable instructions which upon execution by the processor may cause the processor to perform the above-mentioned operations.

FIG. 9 is a diagram that illustrates a set of locations on the scalp of the user for the placement of the one or more electrodes, in accordance with an embodiment of the disclosure. FIG. 9 is explained in conjunction with elements from FIG. 1, FIG. 2, FIG. 3, FIG. 4, FIG. 5, FIG. 6, FIG. 7, and FIG. 8. With reference to FIG. 9, there is shown a diagram 900. The diagram 900 may include an exemplary scalp 902 of the user 114 of FIG. 1. With reference to FIG. 9, there is further shown a nose 904A, a left ear 904B, and a right ear 904C of the user 114. There is further shown a top view 906 of the scalp 902 of the user 114 with a set of positions 908-922 for the placement of the one or more electrodes.

In an embodiment, the user 114 may have to detach the one or more electrodes form the eyeglass 802 and may have to manually place them on at least one of the set of locations 908-922 on their scalp. The set of locations 908-922 may be precisely chosen locations on the scalp for placing the one or more electrodes to ensure consistent and accurate data collection. The set of locations 908-922 may include, but are not limited to, a first location 908, a second location 910, a third location 912, a fourth location 914, a fifth location 916, a sixth location 918, a seventh location 920, and an eighth location 922.

Each location for the placement of the one or more electrodes may have a letter to identify the lobe, or area of the brain of the user 114 it may be reading from. For example, the lobe or the area may be one of a pre-frontal lobe (Fp), a frontal lobe (F), a temporal lobe (T), a parietal lobe (P), an occipital lobe (O), and a central lobe (C). In some embodiments, there may be zero (Z) that may refer to a location on a midline sagittal plane of the scalp 902 (or skull) of the user 114.

The first location 908 of the set of locations 908-922 may correspond to a location in the middle between a nasion (a bridge of the nose 904A) and an inion (a prominent bony structure at the back of the skull) of the user 114. The second location 910 of the set of locations 908-922 may correspond to a location at the midline of the forehead of the user 114 and may be located between the nasion and a hairline of the user 114. The third location 912 of the set of locations 908-922 may correspond to a location on the left side of the scalp 902, approximately at 30% of the distance between the nasion and the inion of the user 114. The third location 912 may be specifically located above a left preauricular point. The fourth location 914 of the set of locations 908-922 may correspond to a location on the right side of the scalp 902, approximately at 30% of the distance between the nasion and the inion of the user 114. The fourth location may be specifically located above a right preauricular point.

The fifth location 916 of the set of locations 908-922 may correspond to a location at the midline of the scalp 902, posterior to a vertex (top of a head of the user 114), and approximately at 50% of the distance between the nasion and the inion. The sixth location 918 of the set of locations 908-922 may correspond to a location at the midline of the scalp 902, posterior to an occipital protuberance (a bony prominence at the base of the skull), and approximately at 50% of the distance between the nasion and the inion. The seventh location 920 of the set of locations 908-922 may correspond to a location on the left side of the scalp 902, approximately at 30% of the distance between the inion and a preauricular point on the left side of the scalp 902. The eighth location 920 of the set of locations 908-922 may correspond to a location on the right side of the scalp 902, approximately at 30% of the distance between the inion and the preauricular point on the right side of the scalp 902.

The user 114 may place the one or more electrodes on at least one of the set of locations 908-922. Once placed, the eyeglass 802 may be configured to control the one or more electrodes to capture one or more brain signals of the user 114. The eyeglass may further detect at least one spike in the captured one or more brain signals and determine one or more actions to be triggered based on the detected at least one spike. The eyeglass 802 may further trigger at least a first action associated with an initiation of a first call as described in FIG. 7, and FIG. 8.

FIG. 10 is a diagram that illustrates an exemplary scenario for alerting users based on brain signals, in accordance with an embodiment of the disclosure. FIG. 10 is explained in conjunction with elements from FIG. 1, FIG. 2, FIG. 3, FIG. 4, FIG. 5, FIG. 6, FIG. 7, FIG. 8, and FIG. 9. With reference to FIG. 10, there is shown an exemplary scenario 1000 of kidnapping of the user 1002. There is further shown a room 1004, an alerting system 1006, a headset 1008, a first user device 1010 associated with a first user 1012.

In the exemplary scenario, the user 1002 may be kidnapped by a kidnapper and may be locked up in the room 1004. The user 114 may not have access to any mobile device and may not be able to alert any authority or other contacts. Due to the nature of the situation, the stress level of the user 1002 may start increasing.

The alerting system 1006 may be configured to control the headset 1008 to capture one or more brain signals associated with a brain of the user 1002. The headset 1008 may capture the one or more brain signals and transmit the captured one or more brain signals to the alerting system 1006. The alerting system 1006 may be further configured to detect at least one spike in the received one or more brain signals. The detected at least one spike may indicate an increase in a stress level of the user 1002. Details about the detection of the at least one spike are provided, for example, in FIG. 4.

The alerting system 1006 may be further configured to determine one or more actions to be triggered based on the detected at least one spike in the received one or more brain signals. The alerting system 1006 may be further configured to control the user device associated with the user 1002 to trigger at least a first action that may be associated with an initiation of a first call, indicating the increased stress level of the user 1002, to the first user device 1010 associated with the first user 1012. The first action may be included in the determined one or more actions.

In another embodiment, the alerting system 1006 may be further configured to control the headset 1008 to trigger at least the first action that may be associated with the initiation of the first call, indicating the increased stress level of the user 1002, to the first user device 1010 associated with the first user 1012. In another embodiment, the alerting system 1006 may be further configured to control the user device associated with the user 1002 and/or the headset 1008 to trigger a second action associated with a transmission of the first message 608 to the first user device 1010 associated with the first user 1012.

FIG. 11 is a flowchart that illustrates an exemplary method for alerting users based on brain waves, in accordance with some embodiment of the disclosure. FIG. 11 is explained in conjunction with elements from FIG. 1, FIG. 2, FIG. 3, FIG. 4, FIG. 5, FIG. 6, FIG. 7, FIG. 8, FIG. 9, and FIG. 10. With reference to FIG. 11, there is shown a flowchart 1100. The operations of the exemplary method may be executed by any computing system, for example, by the alerting system 102 of FIG. 1 or the processor 202 of FIG. 2. The operations of the flowchart 1100 may start at 1102.

At 1102, the alerting system 102 may receive the one or more brain signals from the wearable device 104 worn by the user 114. The received one or more brain signals corresponds an electroencephalography (EEG) signal associated with a brain of the user 114. In at least one embodiment, the processor 202 may be configured to receive the one or more brain signals from the wearable device 104 worn by the user 114, wherein the received one or more brain signals correspond to an electroencephalography (EEG) signal associated with a brain of the user 114. Details about the reception of the one or more brain signals are provided, for example, in FIG. 1, and FIG. 4.

At 1104, at least one spike in the received one or more brain signals may be detected. The detected at least one spike may indicate an increase in a stress level of the user 114. In at least one embodiment, the processor 202 may be configured to detect the at least one spike in the received one or more brain signals, wherein the detected at least one spike indicates the increase in the stress level of the user 114. Details about the detection of the at least one spike are provided, for example, in FIG. 1, and FIG. 4.

At 1106, the one or more actions to be triggered based on the detected at least one spike in the received one or more brain signals may be determined. In at least one embodiment, the processor 202 may be configured to determine the one or more actions to be triggered based on the detected at least one spike in the received one or more brain signals. Details about the determination of the one or more actions are provided, for example, in FIG. 1, and FIG. 4.

At 1108, the user device 106 may be controlled to trigger the at least the first action associated with the initiation of the first call, indicating the increased stress level of the user 114, to the first set of user devices 110 associated with the first set of users 116. In at least one embodiment, the processor 202 may be configured to control the user device 106 to trigger at least the first action associated with the initiation of the first call, indicating the increased stress level of the user 114, to the first set of user devices 110 associated with the first set of users 116, wherein the first action is included in the determined one or more actions. Details about the triggering of the first action are provided, for example, in FIG. 1, FIG. 4, and FIG. 7. Control may pass to end.

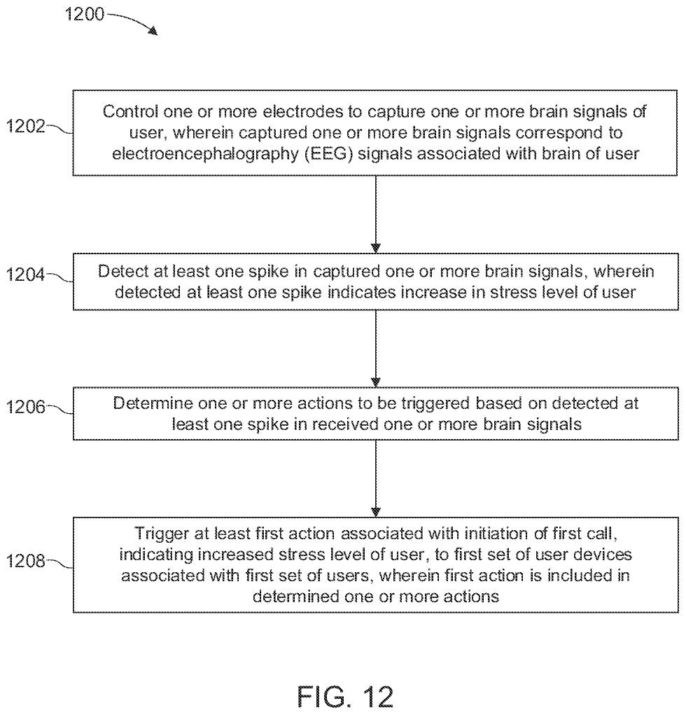

FIG. 12 is a flowchart that illustrates an exemplary method for alerting users based on brain waves using eyeglass, in accordance with some embodiment of the disclosure. FIG. 12 is explained in conjunction with elements from FIG. 1, FIG. 2, FIG. 3, FIG. 4, FIG. 5, FIG. 6, FIG. 7, FIG. 8, FIG. 9, FIG. 10, and FIG. 11. With reference to FIG. 12, there is shown a flowchart 1200. The operations of the exemplary method may be executed by any computing system, for example, by the eyeglass 802 of FIG. 8. The operations of the flowchart 1200 may start at 1202.

At 1202, the one or more electrodes may be controlled to capture the one or more brain signals of the user 114. The captured one or more brain signals correspond to an electroencephalography (EEG) signal associated with a brain of the user 114. The one or more electrodes may be detachably attached to eyeglass 802. In at least one embodiment, the eyeglass 802 may be configured to control the one or more electrodes to capture one or more brain signals of the user 114, wherein the captured one or more brain signals correspond to the electroencephalography (EEG) signal associated with the brain of the user 114. Details about the reception of the one or more brain signals are provided, for example, in FIG. 1, FIG. 4, and FIG. 8.

At 1204, at least one spike in the captured one or more brain signals may be detected. The detected at least one spike may indicate an increase in a stress level of the user 114. In at least one embodiment, the eyeglass 802 may be configured to detect the at least one spike in the received one or more brain signals, wherein the detected at least one spike indicates the increase in the stress level of the user 114. Details about the detection of the at least one spike are provided, for example, in FIG. 1, and FIG. 4.

At 1206, the one or more actions to be triggered based on the detected at least one spike in the received one or more brain signals may be determined. In at least one embodiment, the eyeglass 802 may be configured to determine the one or more actions to be triggered based on the detected at least one spike in the received one or more brain signals. Details about the determination of the one or more actions are provided, for example, in FIG. 1, and FIG. 4.

At 1208, the first action associated with the initiation of the first call may be triggered. The first call may indicate the increased stress level of the user 114 to the first set of user devices 110 associated with the first set of users 116. In at least one embodiment, the eyeglass 802 may be configured to trigger at least the first action associated with the initiation of the first call, indicating the increased stress level of the user 114, to the first set of user devices 110 associated with the first set of users 116, wherein the first action is included in the determined one or more actions. Details about the triggering of the first action are provided, for example, in FIG. 1, FIG. 4, and FIG. 7. Control may pass to end.

Various embodiments of the disclosure may provide a non-transitory computer readable medium and/or storage medium having stored thereon, instructions executable by a machine and/or a computer to operate an alerting system (e.g., the alerting system 102) for alerting users based on brain signals. The instructions may cause the machine and/or computer to perform operations that include controlling one or more processors (e.g., the processor 202). The processor may be configured to receive one or more brain signals from a wearable device (e.g., the wearable device 104) worn by a user (e.g., the user 114). The received one or more brain signals may correspond to an electroencephalography (EEG) signal associated with a brain of the user. The operations may further include detection of at least one spike in the received one or more brain signals. The detected at least one spike may indicate an increase in a stress level of the user. The operations may further include determining one or more actions to be triggered based on the detected at least one spike in the received one or more brain signals. The operations may further include controlling a first user device (e.g., the first user device 110A) to trigger at least a first action associated with an initiation of a first call, indicating the increased stress level of the user, to a first set of user devices (e.g., the first set of user devices 110) associated with a first set of users (e.g., the first set of users 116), wherein the first action is included in the determined one or more actions.

Many modifications and other embodiments of the disclosures set forth herein will come to mind to one skilled in the art to which these disclosures pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

We claim:

1. An alerting system comprising:
a processor;
a memory communicatively coupled to the processor, wherein the memory stores a plurality of processor-executable instructions which upon execution by the processor cause the processor to:
receive one or more brain signals from a wearable device worn by a user, wherein the received one or more brain signals correspond to an electroencephalography (EEG) signal associated with a brain of the user;
detect at least one spike in the received one or more brain signals, wherein the detected at least one spike indicates an increase in a stress level of the user;
determine one or more actions to be triggered based on the detected at least one spike in the received one or more brain signals; and
control a first user device to trigger at least a first action associated with an initiation of a first call, indicating the increased stress level of the user, to a first set of user devices associated with a first set of users, wherein the first action is included in the determined one or more actions.

2. The alerting system of claim 1, wherein the wearable device includes one or more electrodes configured to capture the one or more brain signals of the user.

3. The alerting system of claim 1, wherein the wearable device corresponds to at least one of: a headset, or an eyeglass.

4. The alerting system of claim 3, wherein a first electrode is attached to a first temple tip of the eyeglass and a second electrode is attached to a second temple tip of the eyeglass, and wherein the first electrode and the second electrode is rotatable around the first temple tip and the second temple tip of the eyeglass.

5. The alerting system of claim 1, wherein the wearable device comprises a hardware button, and wherein the processor is further configured to:
receive a first user input via the hardware button included in the wearable device, wherein the first user input corresponds to a selection of the hardware button; and
control the first user device to trigger the first action associated with the initiation of the first call to the first set of user devices based on the reception of the first user input.

6. The alerting system according to claim 1, wherein the initiated first call is a robo-call and transmits a pre-recorded message associated with the detection of the increased stress level of the user to the first set of user devices associated with the first set of users.

7. The alerting system of claim 1, wherein the processor is further configured to:
detect a failure in the initiation of the first call from the first user device to the first set of user devices; and
control the first user device to trigger a second action associated with a transmission of a first message to the first set of user devices, wherein a second priority value associated with the second action is less than a first priority value associated with the first action.

8. The altering system of claim 7, wherein the transmitted first message comprises a timestamp indicative of a time of detection the at least one spike and location information associated with a current location of the user.

9. The alerting system of claim 1, wherein the processor is further configured control the first user device to trigger a third action associated with a transmission of a second message to a second set of user devices associated with a second set of users, wherein the third action is included in the determined one or more actions.

10. The alerting system of claim 1, wherein the processor is further configured to:
control the wearable device to capture the one or more brain signals of the user;
calibrate the captured one or more brain signals, wherein the calibration of the one or more brain signals is specific to the user; and
detect the at least one spike in the received one or more brain signals based on the calibration, wherein the at least one spike is detected based on at least one of: an audio heard by the user, a visual scene seen by the user, a thought of the user, and or a muscular movement of the user.

11. The alerting system of claim 1, wherein the processor is further configured to:
control a first set of wearable devices to capture physiological information associated a set of physiological parameters of the user; and
determine the one or more actions to be triggered based on the detected at least one spike in the received one or more brain signals and the captured physiological information.

12. The altering system of claim 11, wherein the set of physiological parameters comprises a blood pressure rate, a body temperature, a heart rate, a blood oxygen level, and an eye flicker rate.

13. The alerting system of claim 1, wherein the processor is further configured to:
render an electronic user interface (UI) on the first user device, wherein the displayed electronic UI includes a set of UI elements; and
receive, via the set of UI elements, a set of user inputs associated with selection of the first set of users and the one or more actions.

14. The alerting system of claim 1, wherein the processor is further configured to:
compare a value of each of the one or more brain signals with a threshold value stored in a database;
detect the at least one spike in the received one or more brain signals based on the comparison; and
determine, from the database, the one or more actions to be triggered based on the detected at least one spike.

15. A method comprising:
receiving one or more brain signals from a wearable device worn by a user, wherein the received one or more brain signals correspond to an electroencephalography (EEG) signal associated with a brain of the user;
detecting at least one spike in the received one or more brain signals, wherein the detected at least one spike indicates an increase in a stress level of the user;
determining one or more actions to be triggered based on the detected at least one spike in the received one or more brain signals; and
controlling a first user device to trigger at least a first action associated with an initiation of a first call, indicating the increased stress level of the user, to a first set of user devices associated with a first set of users, wherein the first action is included in the determined one or more actions.

16. The method of claim 15, wherein the wearable device corresponds to at least one of: a headset, or an eyeglass.

17. The method of claim 16, wherein a first electrode is attached to a first temple tip of the eyeglass and a second electrode is attached to a second temple tip of the eyeglass, and wherein the first electrode and the second electrode is rotatable around the first temple tip and the second temple tip of the eyeglass.

18. The method of claim 15, wherein the initiated first call is a robo-call and transmits a pre-recorded message associated with the detection of the increased stress level of the user to the first set of user devices associated with the first set of users.

19. The method of claim 15, further comprising:
detecting a failure in the initiation of the first call from the first user device to the first set of user devices; and
controlling the first user device to trigger a second action associated with a transmission of a first message to the first set of user devices, wherein a second priority value associated with the second action is less than a first priority value associated with the first action.

20. A wearable device worn by a user, comprising:
one or more electrodes detachably attached to the wearable device;
a processor;
a memory communicatively coupled to the processor, wherein the memory stores a plurality of processor-executable instructions which upon execution by the processor cause the processor to:
control the one or more electrodes to capture one or more brain signals of the user, wherein the captured one or more brain signals correspond to an electroencephalography (EEG) signal associated with a brain of the user;
detect at least one spike in the captured one or more brain signals, wherein the detected at least one spike indicates an increase in a stress level of the user;
determine one or more actions to be triggered based on the detected at least one spike in the received one or more brain signals; and
trigger at least a first action associated with an initiation of a first call, indicating the increased stress level of the user, to a first set of user devices associated with a first set of users, wherein the first action is included in the determined one or more actions.
